(19) United States
(12) Patent Application Publication
(10) Pub. No.: US 2008/0287733 A1
(43) Pub. Date: Nov. 20, 2008

(54) STIMULATION APPARATUS

(76) Inventor: Donald F. Robinson, Beachwood, OH (US)

Correspondence Address: HOWARD M COHN;PATENT ATTORNEY LLC, 21625 CHAGRIN BLVD #220, CLEVELAND, OH, 44122, US

(21) Appl. No.: 11/748,051

(22) Filed: May 14, 2007

(51) Int. Cl. A61F 5/00; A61N 1/00

(52) U.S. Cl. 600/38; 607/39

(57) ABSTRACT

An apparatus for sexual stimulation in a safe and convenient manner is disclosed. The present invention provides for the in-out motion of an attachment that simulates a penis. The present invention also provides for a vibration mechanism to vibrate the attachment during use. The stroke rate is adjustable by the user. Furthermore, the vibration frequency, and stroke depth are also adjustable by the user during use. The present invention makes use of a mechanical articulating joint, generically known as a spherical rod end or Heim joint for the mounting of an attachment. This provides an additional level of safety and comfort for the user,

### FIELD OF THE INVENTION

This invention relates to sexual aids. More specifically, the present invention relates to powered devices that engage with a user and promote sexual stimulation.

### BACKGROUND

Sexual drive is probably one of the most fundamental and urgent drives that a human experiences. Unfortunately, as the large body of literature, counseling services and devices attest, this drive largely goes unsatisfied. Although psychology plays a large role in attaining satisfaction, physical excitation of the sex organs is a critical consideration

People, for one reason or another, are not always successful in finding partners who satisfy their sexual drive. Some people, especially in view of STDs (sexually transmitted diseases), prefer to abstain rather than engage in human sexual relations. Although sexual relations may be avoided, whether ill-fated or non-disciplined, sexual drive may not. A need exists for an invention that provides for satisfaction of primal sexual drive yet eliminates reliance on human sexual interaction.

### SUMMARY OF THE INVENTION

The present invention provides a sexual stimulation apparatus, comprising: a substantially horizontal base portion:

an elongated arced portion, the arced portion having a cutout area in its top;

two side panels on each end of the elongated arced portion; wherein the arced portion, base portion and the two side panels are disposed to form a housing;

a power supply;

a motor having an output shaft;

a drive assembly;

a vibration unit connected to said drive assembly;

an attachment holder; and

an attachment mounted to said attachment holder in a substantially vertical orientation;

wherein the motor is attached to the drive assembly thereby enabling an up-and-down motion, the attachment holder is attached to the drive assembly via a ball joint, thereby providing for deviation of the attachment from a vertical orientation.

The present invention also provides an apparatus wherein the drive assembly comprises: a plurality of linear bearings;

a support block slidably attached to said plurality of linear bearings:

a drive rail attached to said support block;

a drive bearing disposed to move within said drive rail;

a drive crank having a first and second end, wherein the first end of the drive crank is connected to said bearing by a shaft, and the second end of the drive crank is connected to the output shaft of the motor.

The present invention provides an apparatus wherein the support block is comprised of aluminum.

The present invention provides an apparatus wherein the support block is comprised of plastic.

The present invention also provides an apparatus wherein the support block is comprised of Ultra-High Molecular Weight Polyethylene (UHMW/PE).

The present invention also provides an apparatus wherein the drive rail is comprised of aluminum.

The present invention also provides an apparatus wherein the drive bearing is an annular bearing.

The present invention also provides an apparatus wherein the drive bearing is an ABEC-5 rated bearing.

The present invention also provides an apparatus wherein the drive crank further comprises a slot and an adjustment knob, wherein said shaft traverses the slot and is fastened to the drive crank by the adjustment knob, thereby providing an adjustable stroke depth of the apparatus.

The present invention also provides an apparatus wherein the drive crank further comprises a plurality of measuring marks, thereby providing an indication of the current stroke depth setting.

The present invention also provides an apparatus wherein at least one side panel is secured via a plurality of magnets, thereby facilitating convenient removal of the side panel.

The present invention also provides an apparatus wherein at least one side panel is secured via a hook and loop fastener, thereby facilitating convenient removal of the side panel.

The present invention also provides an apparatus further comprising a control panel configured to control the motor speed, thereby controlling the stroke rate of the apparatus.

The present invention also provides an apparatus further comprising a control panel configured to control the vibration frequency of the vibration unit, thereby controlling the vibration frequency of the apparatus.

The present invention also provides an apparatus further comprising an emergency stop button, thereby providing a means for quickly stopping the apparatus.

The present invention also provides an apparatus having two linear bearings.

The present invention also provides an apparatus wherein the plurality of linear bearings are comprised of stainless steel.

The present invention also provides an apparatus wherein the plurality of linear bearings are approximately 6 inches long.

The present invention also provides an apparatus wherein the plurality of linear bearings are approximately ⅜ inch in diameter.

### BRIEF DESCRIPTION OF THE DRAWINGS

Reference will be made in detail to embodiments of the disclosure, examples of which may be illustrated in the accompanying drawing figures (FIGS.). The figures are intended to be illustrative, not limiting. Although the invention is generally described in the context of these embodiments, it should be understood that it is not intended to limit the invention to these particular embodiments.

Certain elements in selected ones of the figures may be illustrated not-to-scale! for illustrative clarity. The cross-sectional views, if any, presented herein may be in the form of “slices”, or “near-sighted” cross-sectional views, omitting certain background lines which would otherwise be visible in a true cross-sectional view, for illustrative clarity. In some cases, hidden lines may be drawn as dashed lines (this is conventional), but in other cases they may be drawn as solid lines.

If shading or cross-hatching is used, it is intended to be of use in distinguishing one element from another (such as a cross-hatched element from a neighboring un-shaded element. It should be understood that it is not intended to limit the disclosure due to shading or cross-hatching in the drawing figures.

Elements of the figures may (or may not) be numbered as follows The most significant digits (hundreds) of the reference number correspond to the figure number. For example, elements of FIG. 1 are typically numbered in the range of 100-199, and elements of FIG. 2 are typically numbered in the range of 200-299. Similar elements throughout the figures may be referred to by similar reference numerals. For example, the element 199 in FIG. I may be similar (and possibly identical) to the element 299 in FIG. 2. Throughout the figures, each of a plurality of elements 199 may be referred to individually as 199a, 199b, 199c, etc. Such relationships, if any, between similar elements in the same or different figures will become apparent throughout the specification, including, if applicable, in the claims and abstract.

FIG. 1 shows a block diagrams representation of the apparatus of the present invention.

FIG. 2 shows a perspective view of the apparatus of the present invention.

FIGS. 3A and 3B show side views of the apparatus of the present invention.

FIGS. 4A and 4B show details of the drive assembly.

FIG. 5 shows a top view, illustrating major components of the apparatus of the present invention.

### DETAILED DESCRIPTION

The present invention provides an apparatus that provides for sexual stimulation in a safe and convenient manner. The present invention provides for the in-out motion of an attachment that simulates a penis. The present invention also provides for a vibration mechanism to vibrate the attachment during use. The frequency of the in-out motion (referred to as the stroke rate) is adjustable by the user. The vibration frequency and stroke depth (length of travel of the attachment) are also adjustable by the user during use. Another novel feature of the present invention is the use of a mechanical articulating joint, generically known as a spherical rod end or Heim joint for the mounting of the attachment. This provides an additional level of safety and comfort for the user. These and other features and advantages will be described in more detail in reference to the drawings.

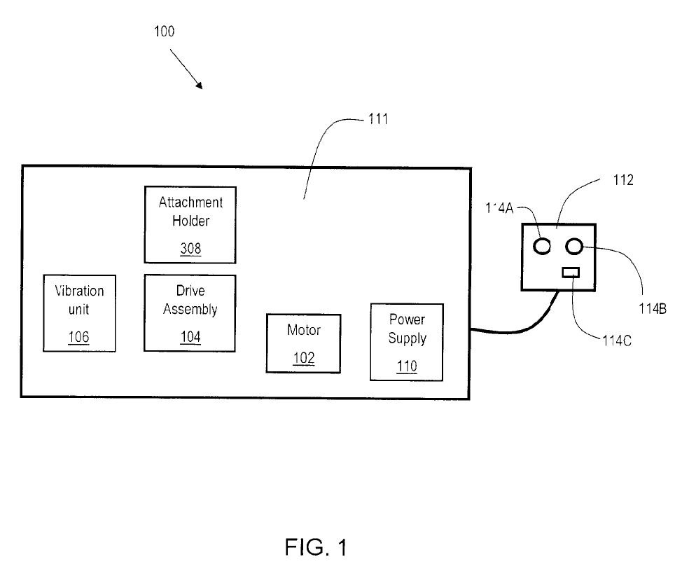

FIG. 1 shows a block diagram representation of the stimulation apparatus I 00 of the present invention. The stimulation apparatus 100 comprises the following major components: A motor 102, a drive assembly 104, a vibration unit 106, an attachment holder 308, and a power supply 110, all contained within a housing 111.

External to the housing 111 is a control panel 112 that controls the apparatus 100. The control panel 112 provides a vibration control 114A and stroke rate control 114B to allow the user to adjust those parameters to achieve optimal comfort and satisfaction. Optionally, an emergency stop button 114C is used to allow the apparatus 100 to be stopped quickly if desired. The control panel is electrically connected to the electrical components within the housing 111 such as the motor 102 and vibration unit 106, as is well understood in this art. In one embodiment the vibration control 114A and stroke rate control 114B are rotary controls with push-button on/off operation. In this way, the controls arc rotated to adjust a parameter, of pushed to quickly enable or disable the corresponding parameter.

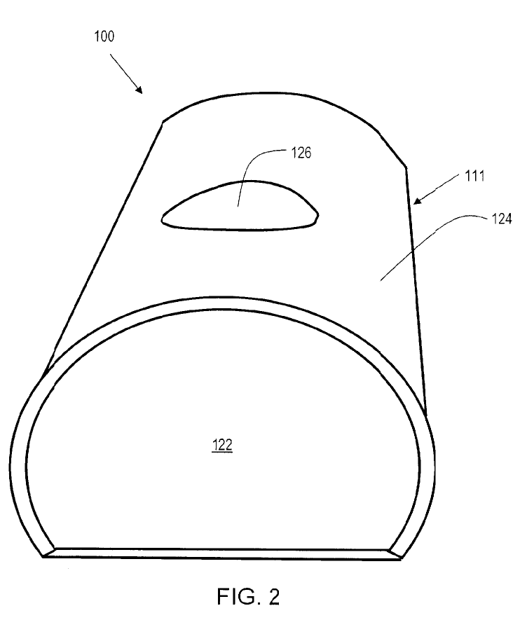

FIG. 2 shows a perspective view of the apparatus I 00 of the present invention. The housing 111 is comprised of an elongated arced portion 124, with a side panel 122 on each side (Only one side panel is shown in FIG. 2). In a preferred embodiment, at least one of the end pieces is quickly removable via a quick-release fastening means, such as magnets, a hook and loop fastener such as VELCRO, or other suitable quick release fastening means. As will be described in more detail later, providing quick access to the internal mechanism of the apparatus provides for a convenient means to adjust the stroke depth. The elongated arced portion 124 has a cutout area 126 on its top to allow the user to have access to the attachment.

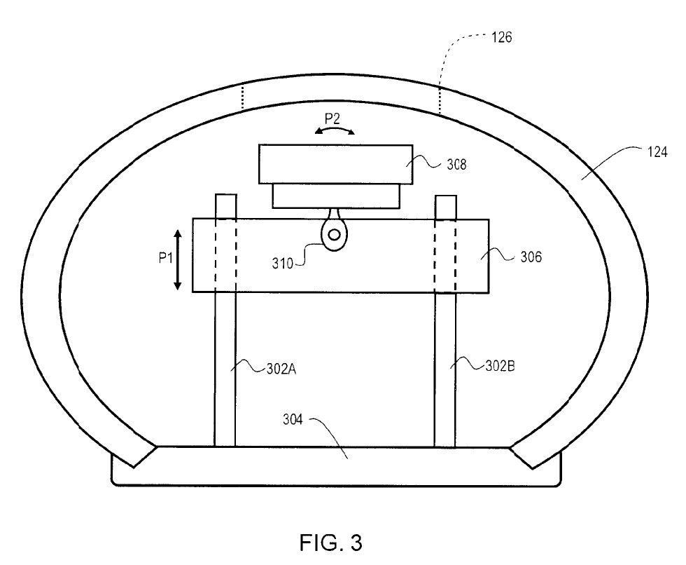

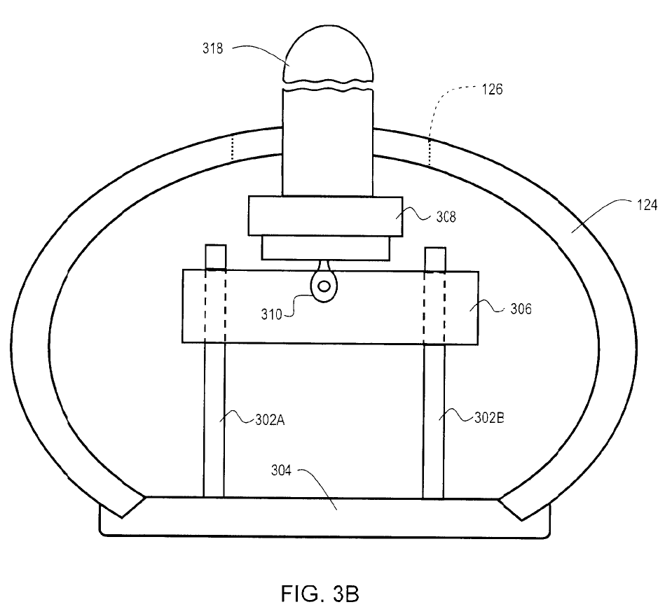

FIG. 3A shows a side view of the apparatus of the present invention. Two linear bearings 3()2A and 302B are supported by base 304, which is substantially horizontal. A support block 306 is slidably attached to the two linear bearings (302A and 302B). In one embodiment, linear bearings (302A, 302B) are THOMSON 440c stainless steel shaft, ⅜ inch diameter, 6 inch long bearings. Suitable bearings are available from McMASTER-CARR corporation.

The block is free to travel along the line indicated by P1. Ball joint 310 is attached to block 306, and supports attachment holder 308. The block is preferably comprised of aluminum or other sturdy, lightweight material. In one embodiment, block 306 is comprised of Ultra-High Molecular Weight Polyethylene (UHMW/PE). Because of its high molecular weight. UHMW/PE has excellent abrasion resistance and a very low coefficient of friction. The ball joint provides a range of allowable motion for the attachment holder 308, so that the attachment has flexibility to tilt a few degrees away from a substantially vertical orientation, as indicated by line P2. The use of a ball joint or mechanical articulating joint provides an additional level of safety and comfort for the user. In one embodiment, the ball joint is a 10−32× 3/16 stainless steel TEFLON lined ball joint. Suitable ball joints are readily available from McMASTER-CARR corporation.

FIG. 3B shows a side view of the apparatus of the present invention as used with an attachment 318.

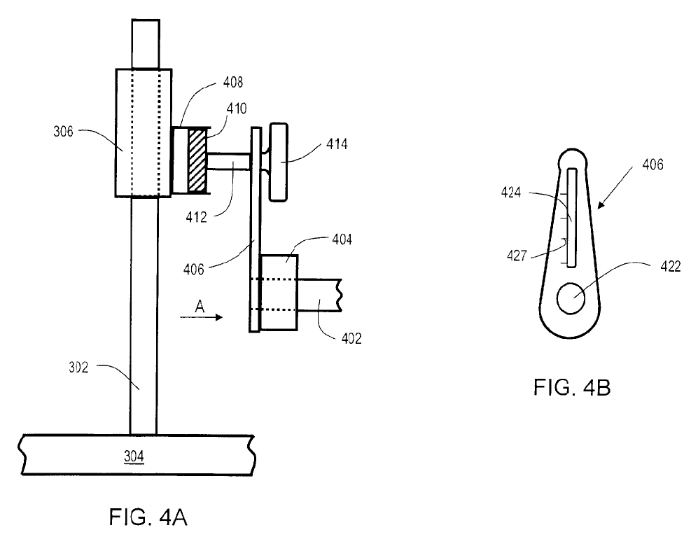

FIGS. 4A and 4B show details of the drive assembly. The base 304 supports two linear bearings (only one is shown in FIG. 4A, indicated as 302). Attached to the linear bearings is block 306. Attached to block 306 is drive rail 408. In one embodiment, the drive rail 408 is comprised of aluminum. However, any suitable material that is sufficiently durable and exhibiting similar frictional properties may be used. The drive rail 408 serves to form a groove allowing a drive bearing to travel therein. Within drive rail 408 a drive bearing 410 is disposed. In one embodiment, the drive bearing is an ABEC-5 rated annular bearing. ABEC (Annular Bearing Engineers' Committee) works to determine the standards for bearings for the Anti-Friction Bearing Manufacturers Association (AFBMA).

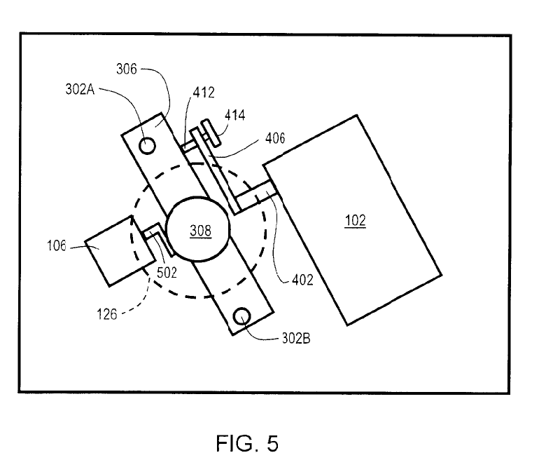

Attached to bearing 410 is a shaft 412 that traverses slot 424 (FIG. 4B) and is fastened to drive crank 406 via stroke adjustment knob 414. Drive crank 406 has mount 422 (FIG. 4B) for receiving the output shalt 402 of the motor (102 in FIG. 5). Optionally, along the length of slot 424 is an indication, such as a plurality of measuring marks indicated generally as 427. When the stroke adjustment knob 414 is aligned with a particular measuring mark the current stroke depth setting is indicated. By adjusting the stroke adjustment knob 414 to align with a different measuring mark, the stroke depth is increased or decreased, thereby providing the user with the ability to adjust the stroke depth for optimal comfort and pleasure.

FIG. 5 shows a top view, illustrating major components of the apparatus of the present invention. Vibration unit 106 is attached to attachment holder 308 via bracket 502. Tie vibrations generated by vibration unit 106 are transmitted to the attachment holder 308, and ultimately to the attachment 318 (FIG. 3B) via bracket 502. The vibration unit is preferably similar to that found in a handheld vibration unit.

Motor 102 has output shaft 402, which is attached to drive crank 406. As output shaft 402 rotates, it moves drive crank 406, resulting in an up-and-down motion (along the line of P1, FIG. 3A) of the attachment holder 308. In one embodiment, the motor 102 is a 62 rpm rated motor that provides 45 inch pounds of torque, and is distributed by GRAINGER as part number 1LPZ5.

The present invention provides an improved apparatus for sexual stimulation. It allows the user real-time control of vibration levels and stroke rate. The stroke depth is also configurable. It will be understood that the present invention may have various other embodiments. Furthermore, while the form of the invention herein shown and described constitutes a preferred embodiment of the invention, it is not intended to illustrate all possible forms thereof. It will also be understood that the words used are words of description rather than limitation, and that various changes may be made without departing from the spirit and scope of the invention disclosed. Thus, the scope of the invention should be determined by the appended claims and their legal equivalents, rather than solely by the examples given.

What is claimed is:

1. A sexual stimulation apparatus, comprising:
a substantially horizontal base portion;
an elongated arced portion, the arced portion having a cutout area in its top;
two side panels on each end of the elongated arced portion; wherein the arced portion, base portion and the two side panels are disposed to form a housing;
a power supply;
a motor having an output shaft;
a drive assembly;
a vibration unit connected to said drive assembly;
an attachment holder; and
an attachment mounted to said attachment holder in a substantially vertical orientation;
wherein the motor is attached to the drive assembly thereby enabling an up-and-down motion, the attachment holder is attached to the drive assembly via a ball joint, thereby providing for deviation of the attachment from a vertical orientation.

2. The apparatus of claim 1, wherein the drive assembly comprises:
a plurality of linear bearings;
a support block slidably attached to said plurality of linear bearings;
a drive rail attached to said support block;
a drive bearing disposed to move within said drive rail;
a drive crank having a first and second end, wherein the first end of the drive crank is connected to said bearing by a shaft, and the second end of the drive crank is connected to the output shaft of the motor.

3. The apparatus of claim 2, wherein the support block is comprised of aluminum.

4. The apparatus of claim 2, wherein the support block is comprised of plastic.

5. The apparatus of claim 4, wherein the support block is comprised of (UHMW/PE).

6. The apparatus of claim 2, wherein the drive rail is comprised of aluminum.

7. The apparatus of claim 2, wherein the drive bearing is an annular bearing.

8. The apparatus of claim 7, wherein the drive bearing is an ABEC-5 rated bearing.

9. The apparatus of claim 2, wherein the drive crank further comprises a slot and an adjustment knob, wherein said shaft traverses the slot and is fastened to the drive crank by the adjustment knob, thereby providing an adjustable stroke depth of the apparatus.

10. The apparatus of claim 9, wherein the drive crank further comprises a plurality of measuring marks, thereby providing an indication of the current stroke depth setting.

11. The apparatus of claim 1, wherein at least one side panel is secured via a plurality of magnets, thereby facilitating convenient removal of the side panel.

12. The apparatus of claim 1, wherein at ]east one side panel is secured via a hook and loop fastener, thereby facilitating convenient removal of the side panel.

13. The apparatus of claim 1, further comprising a control panel configured to control the motor speed, thereby controlling the stroke rate of the apparatus.

14. The apparatus of claim 13, further comprising a control panel configured to control the vibration frequency of the vibration unit, thereby controlling the vibration frequency of the apparatus.

15. The apparatus of claim 14, further comprising an emergency stop button, thereby providing a means for quickly stopping the apparatus.

16. The apparatus of claim 2, wherein the plurality of linear bearings is two linear bearings.

17. The apparatus of claim 2, wherein the plurality of linear bearings are comprised of stainless steel.

18. The apparatus of claim 2, wherein the plurality of linear bearings are approximately 6 inches long.

19. The apparatus of claim 18, wherein the plurality of linear bearings are approximately ⅜ inch in diameter.

20. A sexual stimulation apparatus, comprising:
a substantially horizontal base portion;
an elongated arced portion, the arced portion having a cutout area in its top;
two side panels on each end of the elongated arced portion; wherein the arced portion, base portion and the two side panels are disposed to form a housing;
a power supply;
a motor having an output shaft;
a drive assembly comprising a support block:
a vibration unit connected to said drive assembly;
an attachment holder; and
an attachment mounted to said attachment holder in a substantially vertical orientation;
wherein the motor is attached to the drive assembly thereby enabling an up-and-down motion, the attachment holder is attached to the support block via a ball joint, thereby providing for deviation of the attachment from a vertical orientation;
wherein the drive assembly comprises:
two linear bearings;
a support block slidably attached to the linear bearings;
a drive rail attached to said support block;
a drive bearing disposed to move within said drive rail;
a drive crank having a first and second end, wherein the first end of the drive crank is connected to said bearing by a shaft, and the second end of the drive crank is connected to the output shaft of the motor;
wherein the drive crank further comprises a slot and an adjustment knob, wherein said shaft traverses the slot and is fastened to the drive crank by the adjustment knob thereby providing an adjustable stroke depth of the apparatus; and
wherein at least one side panel is secured via a plurality of magnets, thereby facilitating convenient removal of the side panel.
